(19) United States
(12) Patent Application Publication
(10) Pub. No.: US 2026/0278285 A1
(43) Pub. Date: Sep. 17, 2026

(54) SYSTEM, DEVICE, AND METHOD OF SIMULATING COMPUTER INPUT COMMANDS TO GENERATE RADIOLOGY REPORTS BASED ON SPEECH

(71) Applicant: PolyRad, Inc., Los Angeles, CA (US)

(72) Inventor: Dane Alexander Weinert, Los Angeles, CA (US)

(21) Appl. No.: 19/078,110

(22) Filed: Mar. 12, 2025

(51) Int. Cl. G06F 40/40; G06F 3/023; G16H 15/00

(52) U.S. Cl. CPC G06F 40/40; G06F 3/023; G16H 15/00

(57) ABSTRACT

The present disclosure describes a device for simulating keyboard input. It includes an electronic processing unit, a microcontroller, IO ports, a microphone, an activator, and a software application. The first IO port connects to a computer. The software uses large language models (LLMs) for various tasks: a first LLM for speech-to-text conversion, a second LLM for generating a fast report, and a third LLM for refining the report. The microcontroller generates keystrokes based on the refined report and outputs them through the first IO port. The software converts audio input to text, then to a fast report, and finally to a refined report. It also processes macros to modify the texts. This device enables efficient and accurate keyboard input simulation based on audio and macros.

### FIELD OF USE

The present disclosure relates generally to the field of inputting information into a computer. More specifically, the present disclosure relates to a device that may receive shorthand oral information and translate the information into a radiology report style write-up.

### BACKGROUND

In the field of medical diagnostics, radiology plays a crucial role in identifying and treating various conditions through imaging techniques, such as, but not limited to, X-rays, CT (computed tomography) scans, MRI (magnetic resonance imaging) scans, and ultrasounds. Radiologists are responsible for interpreting these images and generating detailed reports that summarize their findings. These reports are essential for guiding patient care and treatment decisions. However, the process of creating radiology reports is often time-consuming and requires an elevated level of accuracy and detail.

Traditionally, radiologists dictate their observations and findings into a voice recorder or directly into a speech recognition system, which then transcribes the spoken words into text. This text must be reviewed and edited to ensure it accurately reflects the radiologist's observations and adheres to the required format and terminology. Despite advancements in speech recognition technology, the transcription process is still prone to errors, and the need for manual review and correction remains.

The increasing workload and demand for radiological services have highlighted the need for more efficient methods of generating radiology reports. Automating the transcription process and reducing the time spent on report generation can significantly enhance productivity and allow radiologists to focus more on image analysis and patient care.

Recent developments in natural language processing (NLP) and machine learning have opened new possibilities for improving the accuracy and efficiency of medical transcription. These technologies can be leveraged to create systems that not only transcribe spoken words but also understand the context and structure of radiology reports, ensuring that the final output is both accurate and professionally formatted.

Thus, what is needed is a system designed to receive shorthand oral information from radiologists and translate it into a structured radiology report.

### SUMMARY OF THE INVENTION

To minimize the limitations in the cited references, and to minimize other limitations that will become apparent upon reading and understanding the present specification, the system for transcribing radiology reports herein relates to a device that connects to a computer that receives audio input and outputs keystrokes based on multiple language models.

In the following description, certain terminology is used to describe certain features of the various embodiments of the device, method, and/or system. For example, as used herein, the terms “computer” and “computer system” generally refer to any device that processes information with an integrated circuit chip.

In a preferred embodiment, all hardware components are contained in, and all steps are performed on, a single device. In alternate embodiments, hardware components may be contained on different devices, and/or steps may be performed on different devices.

In one embodiment, this device significantly enhances the efficiency and accuracy of generating radiology reports. Traditionally, radiologists spend a substantial amount of time transcribing their spoken observations into detailed reports. By leveraging this device, radiologists may dictate their findings in shorthand speech, which the device then translates into comprehensive and grammatically correct reports. This not only speeds up the reporting process but also allows radiologists to focus more on patient care and diagnosis rather than administrative tasks. The automation of report generation reduces the likelihood of human error in transcription, ensuring that critical medical information is accurately communicated.

In another embodiment, the device could be integrated with existing radiology information systems (RIS) and picture archiving and communication systems (PACS), streamlining the workflow within radiology departments. This integration may mean that as soon as a radiologist finishes dictating their observations, the device can immediately generate a full report and store it within the patient's digital records. This seamless process minimizes delays in report availability, which can be crucial for timely patient treatment. Moreover, the consistent use of proper punctuation, grammar, and medical terminology enhances the clarity and professionalism of the reports, which is essential for interdisciplinary communication and for maintaining exacting standards of documentation within the medical field.

In one embodiment, the device employs advanced natural language processing (NLP) algorithms and machine learning techniques to continually improve its accuracy and adaptability to individual radiologists' speech patterns. Over time, the system can learn specific preferences and terminologies used by different radiologists, further personalizing the report generation process. This adaptability ensures that the final reports are not only accurate but also tailored to the specific style and requirements of each user, thereby promoting user satisfaction, and increasing adoption rates. Additionally, the use of such cutting-edge and innovative technology (1) keeps the radiology department at the forefront of medical innovation and (2) betters patient outcomes through improved diagnostic efficiency.

In one embodiment,

[symbol]

The various features and embodiments may be combined in multiple combinations so that any feature in the disclosure may be present in any combination of disclosure elements.

Other features and advantages inherent in the disclosed system and method for delivery metric analysis and notification, besides those which are claimed and disclosed, will become apparent to those skilled in the art from the following detailed description and its accompanying drawings.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps, which are illustrated. When the same numeral appears in different drawings, it refers to the same or like components or steps.

FIG. 1 is a block diagram of one embodiment of a device for simulating keyboard input.

FIG. 2 is a screenshot showing one embodiment of a user interface of the software application.

FIG. 3 is a screenshot showing one embodiment of a user interface of the software application.

FIG. 4 is a screenshot showing one embodiment of a user interface of the software application.

FIG. 5 is a screenshot showing one embodiment of a user interface of the software application.

FIG. 6 is a screenshot showing one embodiment of a user interface of the software application.

FIG. 7 is a screenshot showing one embodiment of a user interface of the software application.

FIG. 8 is a screenshot showing one embodiment of a user interface of the software application.

FIG. 9 is a screenshot showing one embodiment of a user interface of the software application.

FIG. 10 is a screenshot showing one embodiment of a user interface of the software application.

FIG. 11 is a screenshot showing one embodiment of a user interface of the software application.

FIG. 12 is a screenshot showing one embodiment of a user interface of the software application.

FIG. 13 is a screenshot showing one embodiment of a user interface of the software application.

FIG. 14 is a screenshot showing one embodiment of a user interface of the software application.

FIG. 15 is a screenshot showing one embodiment of a user interface of the software application.

FIG. 16 is a screenshot showing one embodiment of a user interface of the software application.

FIG. 17 is a screenshot showing one embodiment of a user interface of the software application.

FIG. 18 is a screenshot showing one embodiment of a user interface of the software application.

FIG. 19 is a screenshot showing one embodiment of a user interface of the software application.

FIG. 20 is a screenshot showing one embodiment of a user interface of the software application.

FIG. 21 is a screenshot showing one embodiment of a user interface of the software application.

FIG. 22 is a screenshot showing one embodiment of a user interface of the software application.

FIG. 23 is a screenshot showing one embodiment of a user interface of the software application.

FIG. 24 is a screenshot showing one embodiment of a user interface of the software application.

FIG. 25 is a screenshot showing one embodiment of a user interface of the software application.

FIG. 26 is a screenshot showing one embodiment of a user interface of the software application.

FIG. 27 is a screenshot showing one embodiment of a user interface of the software application.

FIG. 28 is a screenshot showing one embodiment of a user interface of the software application.

FIG. 29 is a screenshot showing one embodiment of a user interface of the software application.

FIG. 30 is a screenshot showing one embodiment of a user interface of the software application.

FIG. 31 is a screenshot showing one embodiment of a user interface of the software application.

FIG. 32 is a flow block diagram of one embodiment of a system for simulating keyboard input.

FIG. 33 is a flow block diagram of one embodiment of a system and method for simulating keyboard input.

FIG. 34 is a flow block diagram of one embodiment of a system and method for processing speech to simulate keyboard input.

### DETAILED DESCRIPTION OF THE DRAWINGS

In the following detailed description, numerous specific details are set forth in order to provide a thorough understanding of various aspects of one or more embodiments. However, these embodiments may be practiced without some or all of these specific details. In other instances, well-known methods, procedures, and/or components have not been described in detail so as not to unnecessarily obscure aspects of embodiments.

While multiple embodiments are disclosed, still other embodiments will become apparent to those skilled in the art from the following detailed description. As will be realized, these embodiments are capable of modifications in various obvious aspects, all without departing from the spirit and scope of protection. Accordingly, figures and the detailed descriptions thereof are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about,” may refer to a deviance of between 0.0001-10% from the indicated number or range of numbers.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

As will be appreciated by one skilled in the art, the systems and methods may take the form of an entirely hardware embodiment, an entirely software embodiment, or an embodiment combining software and hardware embodiments. Furthermore, the systems and methods may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, compact discs, read-only-memory (CD-ROMs), optical storage devices, or magnetic storage devices.

Embodiments of the systems and methods are described below with reference to schematic diagrams, block diagrams, and flowchart illustrations of methods, systems, apparatuses, and computer program products. It will be understood that each block of the block diagrams, schematic diagrams, and flowchart illustrations, and combinations of blocks in the block diagrams, schematic diagrams, and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of the various embodiments of the device, method, and/or system. For example, as used herein, the terms “computer” and “computer system” generally refer to any device that processes information with an integrated circuit chip and/or central processing unit (CPU).

As used herein, the terms “software” and “application” refer to any set of machine-readable instructions on a machine, web interface, and/or computer system” that directs a computer's processor to perform specific steps, processes, or operations disclosed herein.

As used herein, the term “computer-readable medium” refers to any storage medium adapted to store data and/or instructions that are executable by a processor of a computer system. The computer-readable storage medium may be a computer-readable non-transitory storage medium and/or any non-transitory data storage circuitry (e.g., buggers, cache, and queues) within transceivers of transitory signals. The computer-readable storage medium may also be any tangible computer readable medium. In various embodiments, a computer-readable storage medium may also be able to store data, which is able to be accessed by the processor of the computer system.

As used herein, the terms “device”, “computer”, “computer system”, “electronic data processing unit”, and “server” refer to any device that processes information with an integrated circuit chip, including without limitation, personal computers, mainframe computers, workstations, servers, desktop computers, portable computers, laptop computers, embedded computers, wireless devices including cellular phones, personal digital assistants, tablets, tablet computers, smart phones, portable game players, wearables, smart devices and hand-held computers. The term “internet” refers to any collection of networks that utilizes standard protocols, whether Ethernet, Token ring, Wi-Fi, asynchronous transfer mode (ATM), Fiber Distributed Data Interface (FDDI), code division multiple access (CDMA), global systems for mobile communications (GSM), long term evolution (LTE), or any combination thereof. The term “website” refers to any document written in a mark-up language including, but not limited to, hypertext mark-up language (HTML) or virtual reality modeling language (VRML), dynamic HTML, extended mark-up language (XML), wireless markup language (WML), or any other computer languages related thereto, as well as to any collection of such documents reachable through one specific Internet Protocol Address or at one specific World Wide Web site, or any document obtainable through any particular Uniform Resource Locator (URL). Furthermore, the terms “webpage,” “page,” “website,” or “site” refers to any of the various documents and resources on the World Wide Web, in HTML/XHTML format with hypertext links to enable navigation from one page or section to another, or similar such resources used on the Internet.

As used herein, the term “sender” refers to the valid author or transmitting user of a share, digital asset, file, link, content, or resource from their mobile or fixed computing device. The sender configures the asset, and its security context factors, as well as chooses the parameters including timing, synchronicity, lifespan, access payment/fee and target recipients as well as the method or mode of transmission.

As used herein, the term “recipient” refers to the valid peer receiver, consumer, purchaser or engaging user of a sender's share, digital asset, file, link, content, or resource on their mobile computing device. The recipient accesses a share on their device from any possible engagement channel and is authenticated and authorized via peer payment to the sender by the present device, system, and/or method according to their local and share global fees and policies set by the sender. The recipient is allowed, denied, or misdirected access to the share source content or asset based on passage or failure to authenticate in context, execute the peer payment for the access or any other parameter required or set by the sender. The recipient can, in turn, become their own sender/seller of the same or as new content from their mobile device to other recipients. There may be one or more recipients.

As used herein, the terms “share” or “asset” refer to one or more, or a collection of one or more, encrypted or unencrypted hyperlinks, digital files, documents, images, sound or video files, streams, payments, events, downloads, locations or directions, communication, messages, tweets, posts, emails, short codes, apps or games or game events, knowledge, performance or other electronic manifestation of content capable of being authored, saved, stored, hosted, referenced, downloaded, transmitted and accessed, consumed, opened or otherwise engaged from a computing device according to the steps of the present method.

As used herein, the terms “secured share” or “secured asset” refer to an encrypted or unencrypted asset, file, or hyperlink that offers abstraction and indirection from the original protected source file, link, or asset and is used to transmit from sender to user in lieu of the original asset. The secured share link calls up the system processing when clicked or tapped on a mobile computing device for purposes of allowing or denying access according to the share and user context.

The terms “host” or “platform” refers to any computer network, platform, website or app infrastructure for public or private registration, connection, communication, hosting, sharing, streaming, access or presentation/publication of content, messages, information, data, files, streams, media, documents or computer code by users accessing from either fixed or mobile computing devices. Such “hosts” or “platforms” may include, but are not limited to, popular networks such as Facebook®, Instagram®, Yahoo®, Twitter®, Dropbox®, Box®, Evernote®, Apple® iCloud®, iMessage®, Google Cloud®, Microsoft® OneDrive®, SkyDrive®, Skype®, public email, text messaging, SMS, a web server, an app server, a mobile apps, wearable platforms, local networks and storage, chat or instant messaging.

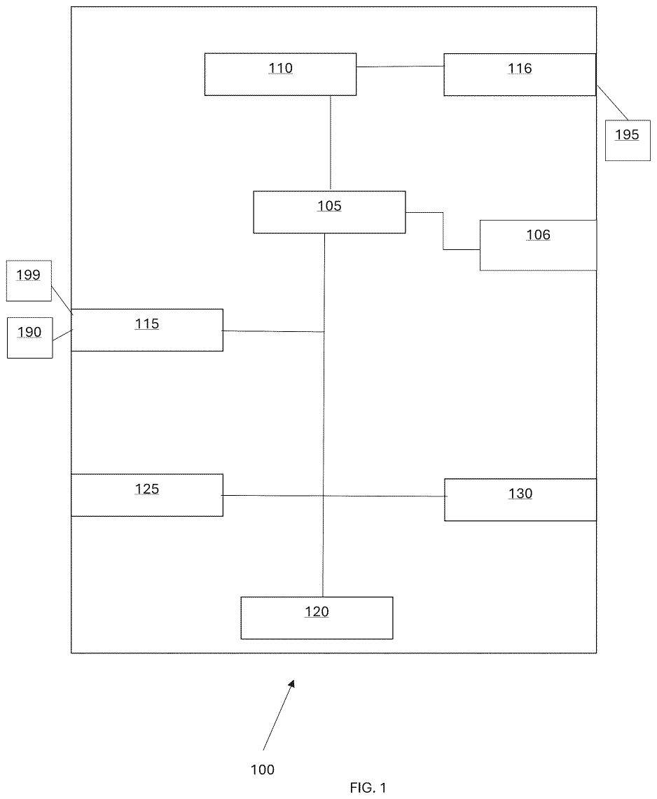

FIG. 1 is a diagram of one embodiment of a device for simulating keyboard input 100. As shown in FIG. 1, device 100 may comprise electronic processing unit (“EPU”) 105, power source 106, which may preferably be an A/C power cord or a battery, microcontroller 110, one or more IO ports 115, 116, software application 120, which may be more than one application, microphone 125, and activator 130. In one embodiment, audio may be processed by device 100 and the output may be a radiology report that is output by mimicking keyboard keystrokes.

EPU 105 may serve as the central processing unit of the device. It may be responsible for executing software application 120, managing data flow between components, and performing complex computations required for speech recognition, translation, and text generation. The EPU 105 may include one or more processors, such as a central processing unit (CPU), a graphics processing unit (GPU), and/or specialized artificial intelligence (AI) processors (including one or more generative AI systems and/or one or more large language model (LLM) systems) designed and configured to handle machine learning tasks efficiently.

Microcontroller 110 may act as an intermediary between EPU 105 and other hardware components. Microcontroller 110 may receive text, or other types of interface input from EPU 105 and output that text input through IO ports 115, 116, as necessary. In one embodiment, IO port 115 may connect to workstation 195, which may accept the rendered “typing” from microcontroller 110 provided as a simulation of a “keyboard” interface.

IO ports 115, 116 may provide interfaces for connecting external devices, such as device 195, external peripherals, such as external peripheral 199, and/or the cloud/external databases, such as database/cloud 190. IO ports 115, 116 may have or include USB ports, audio jacks, and other standard connectors. IO port 115 may facilitate the input of spoken audio through an external microphone 199 in embodiments where microphone 125 is not integrated into device 100. In one embodiment, IO port 116 may output the final radiology text as keyboard strokes to a connected computer workstation 195 or to other devices. In one embodiment, IO port 115 may be a connection for the configuration of the software as provided by database/cloud 190.

Software application 120 may include a speech recognition module that converts spoken audio into text, which may be a speech to text LLM, a translation module that processes the text through one or more LLMs, and a text generation module that formats the final radiology text. The software application 120 may also include a keyboard emulation module that outputs the final text as keyboard strokes to the connected device 195 through IO port 115. In one embodiment, the LLMs may comprise three (3) LLMs, a first LLM that comprises a speech to text protocol, a second LLM that comprises a fast output protocol, and a third LLM that comprises a refined output protocol.

Microphone 125 may be an audio input device that captures spoken audio from a user. It may be designed to pick up clear and high-quality audio signals to ensure accurate speech recognition. Microphone 125 may include noise-canceling features to minimize background noise and enhance the clarity of the captured audio.

Activator 130 may be a user interface component that allows the user to activate or deactivate the device. It may be a physical button or switch, a touch-sensitive area, or a voice-activated command. Activator 130 may ensure that the device only processes audio when explicitly instructed by the user, preventing unintended processing.

In one embodiment, an operational flow for converting spoken words to a radiology text output may be as follows.

First, the user may speak into microphone 125, which may capture the spoken audio and send it for processing to the EPU 105, where a speech recognition module or first LLM of software application 120 may convert the audio into text.

The text may then be processed through the second LLM, and then the third LLM. Each LLM may provide different perspectives or enhancements to the text, ensuring a comprehensive and accurate translation.

The outputs from the three LLMs may preferably produce the final radiology text. In one embodiment, the first LLM is configured to convert the audio to a transcription text. In one embodiment, the second LLM is configured to convert the transcription text to a fast report text. In one embodiment, the third LLM is configured to convert the fast report text to a refined report text. The refined report text may be considered a final radiology text.

In some embodiments the multiple LLMs may be combined into fewer LLMs. In some embodiments, this division of functionality between LLMs may allow for an overall faster or less energy intensive outputs. For example, the first LLM may simply convert speech to text, the second LLM may convert specific text to processed text based on pre-loaded macros, and the third LLM may convert the processed text to refined text utilizing higher functioning LLM protocols.

The final radiology text may be sent to the keyboard emulation module, which may be the microcontroller 110, which may convert the processed and finalized text into keyboard strokes.

The keyboard strokes may be sent through the IO port 116 to the connected device, such as workstation 195, where the text may be inputted into workstation 195 as if it were typed by a physical keyboard. In some embodiments, IO ports 115, 116 may be wired or wireless.

In some embodiments, device 100 may connect to database 190, which may be within a computer or server, wherein the user may use that computer or server to interact with a software application, which may or may not include software application 120. In some embodiments, the remotely located software application may comprise a user interface that may facilitate the user providing instructions or inputting macros. In some embodiments, these macros may be implemented into one or more of the LLMs to allow the LLMs to process the audio in a way customized to the user. In one example, the user may input a macro that causes a specific word or shorthand to be replaced in one or more of the text outputs with something else. In some embodiments, the software application may comprise customizable LLM parameters, prompts, templates, macros, impressions, classifications, and fine-tuned LLMs themselves (fine-tuned to the user's style via speech-report training pairs). In some embodiments, the software application may also learn based on repeated audio inputs that are attempting to clarify or reach a particular output.

In one example, device 100 may be configured to, but is not limited to, process the following audio of:
“6 cm liver mass increased from prior series image 69, new hyperenhancing segment 6 mass, steatosis, mild wall thickening gallbladder otherwise normal”

and the output text on the screen may be as follows:

### “FINDINGS:

Visualized Lower Thorax: Limited imaging of the thorax demonstrates no suspicious pleural or parenchymal disease. Visualized heart is unremarkable.
Liver: The liver has a large 6 cm mass, increased in size from prior exam (series 4 image 69), as well as a new hyper-enhancing mass in segment 6. There is hepatic steatosis. Portal veins are patent.
Gallbladder: The gallbladder has mild wall thickening without pericholecystic fluid or stones.
Biliary System: No intrahepatic or extrahepatic bile duct dilatation.
Spleen: Unremarkable.
Pancreas: Unremarkable without definite focal lesion. No pancreatic ductal dilatation.
Adrenal Glands: Unremarkable.
Kidneys/Urinary Tract: The kidneys are unremarkable in morphology bilaterally with symmetric renal enhancement. No suspicious renal mass. No hydronephrosis. The ureters, as visualized, are normal in course and caliber. The bladder is within normal limits.
Pelvis: Unremarkable.
GI Tract: The bowel, as visualized, is within normal limits. A normal appendix is identified.
Vasculature: Abdominal aorta is normal in caliber.
Lymph Nodes: No enlarged lymph nodes in the abdomen or pelvis.
Intraperitoneal/Retroperitoneal Space: No free air or fluid in the abdomen or pelvis.
Musculoskeletal/Soft Tissue: No acute osseous abnormality. No suspicious osseous lesions. No acute soft tissue abnormality.

IMPRESSION:
1. Multiple liver masses, one of which is increased in size and one of which is new from prior exam, concerning for metastatic disease.
2. No evidence of acute cholecystitis.
3. Hepatic steatosis.”

In other embodiments, particular dictations may result in different text output, depending on the user's preferences, settings, and templates.

In some embodiments, the user may provide instructions or macros to the software application to allow the conversion of speech to text with specific changes, as preferably processed by the LLMs.

In some embodiments, EPU 105 may be substantially any commercially available CPU or GPU, or equivalents thereof.

In one embodiment, EPU 105 may be a Jetson Orin AGX. In one embodiment, microcontroller 110 may be an ESP32-S3 micro-controller.

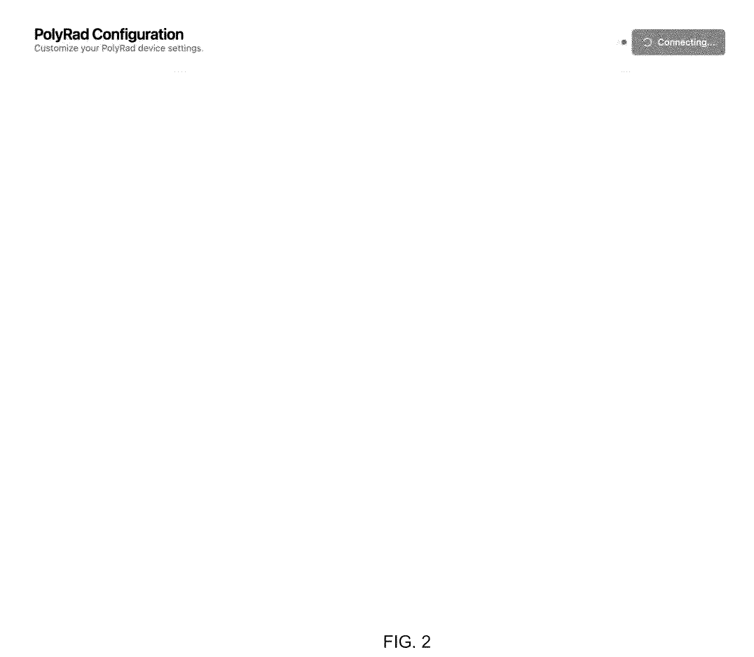

FIG. 2 is a screenshot showing one embodiment of a user interface of the software application. As shown in FIG. 2, the user interface may comprise a connecting screen.

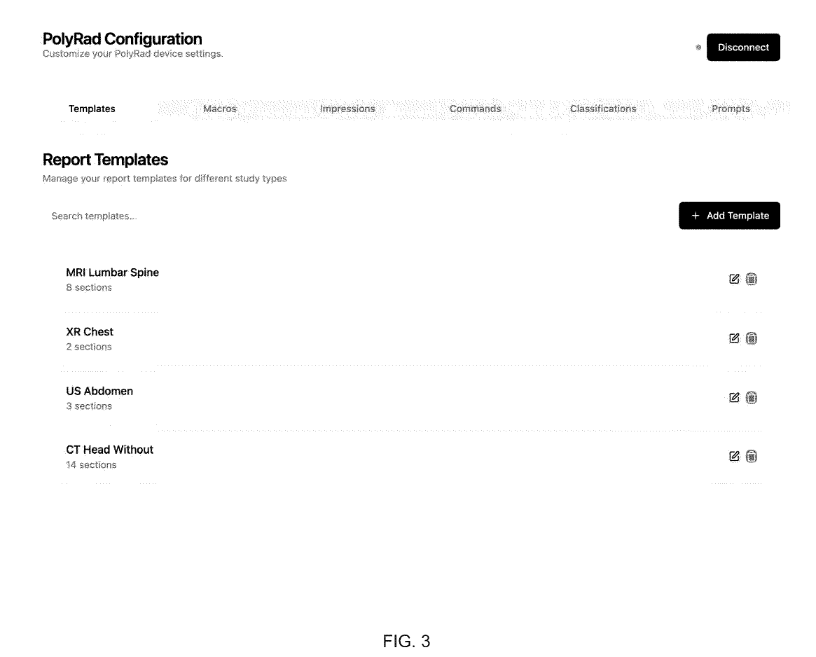

FIG. 3 is a screenshot showing one embodiment of a user interface of the software application. As shown in FIG. 3, the user interface may comprise a report templates screen.

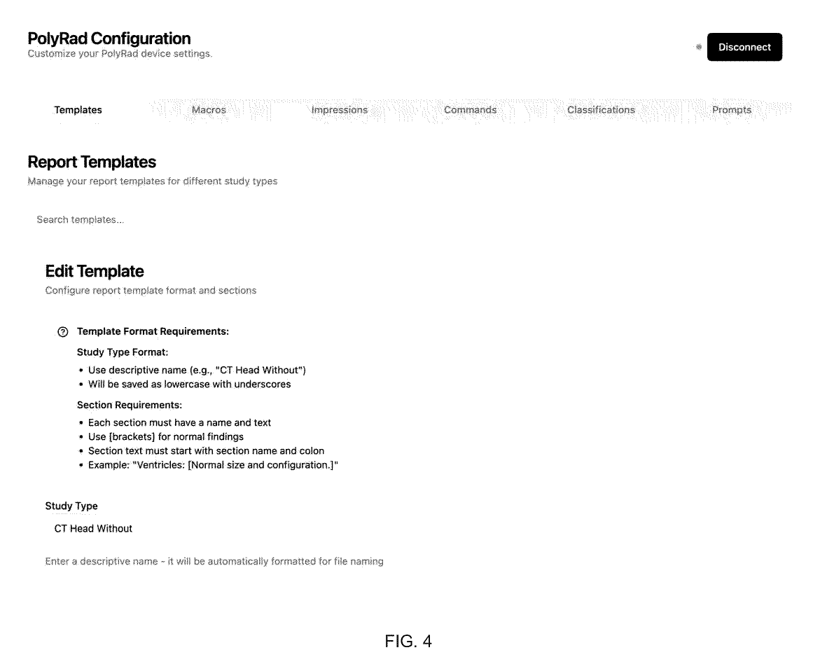

FIG. 4 is a screenshot showing one embodiment of a user interface of the software application. As shown in FIG. 4, the user interface may comprise an edit templates screen.

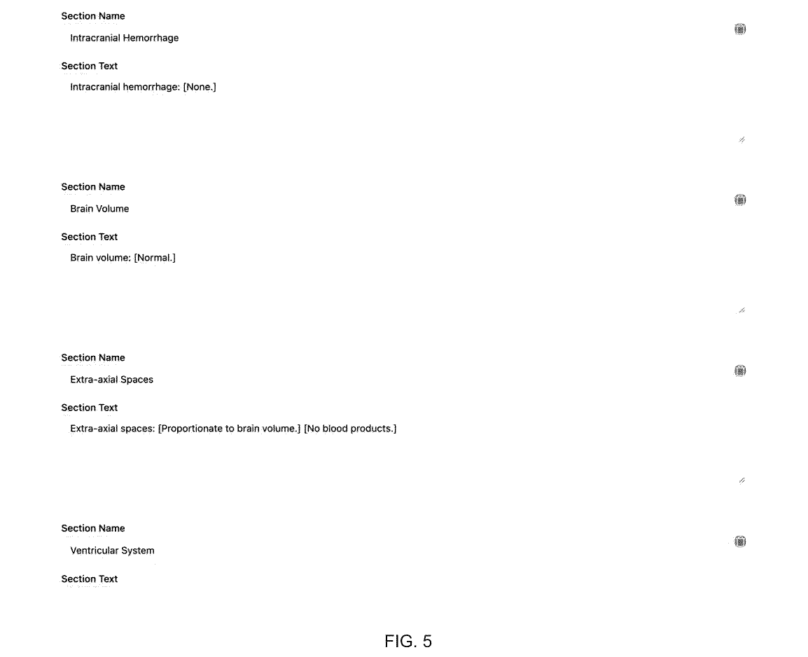

FIG. 5 is a screenshot showing one embodiment of a user interface of the software application. As shown in FIG. 5, the user interface may comprise an editing templates screen showing the section name and text.

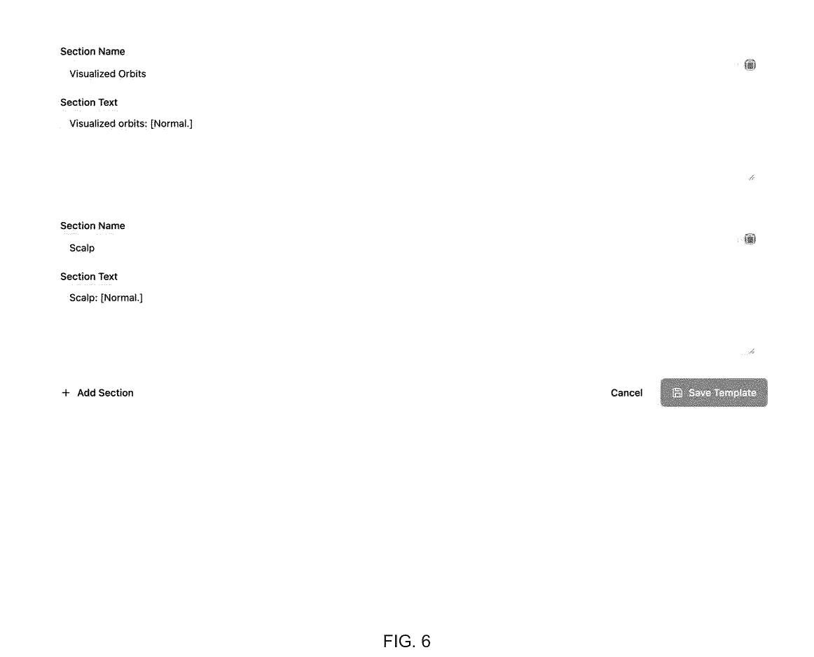

FIG. 6 is a screenshot showing one embodiment of a user interface of the software application. As shown in FIG. 6, the user interface may comprise a save button for editing the templates.

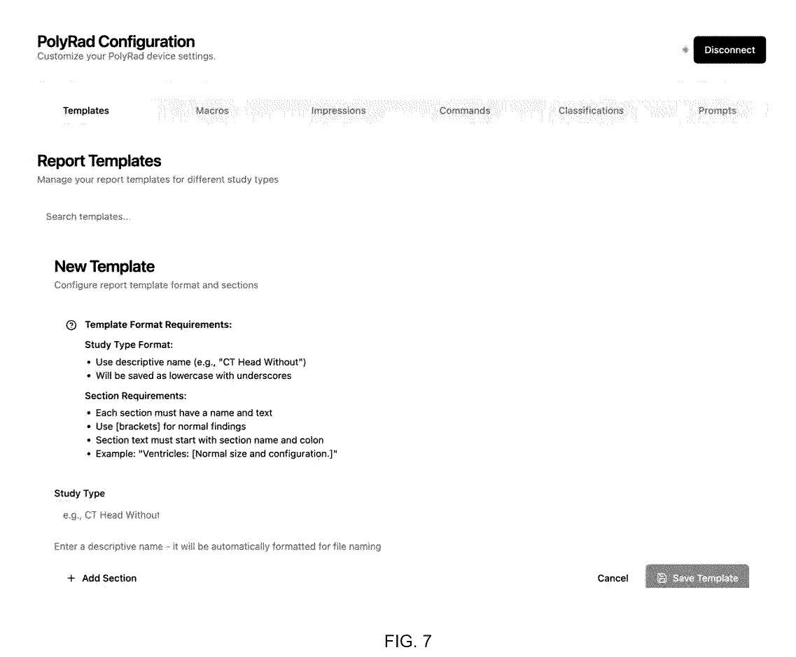

FIG. 7 is a screenshot showing one embodiment of a user interface of the software application. As shown in FIG. 7, the user interface may comprise a new template screen.

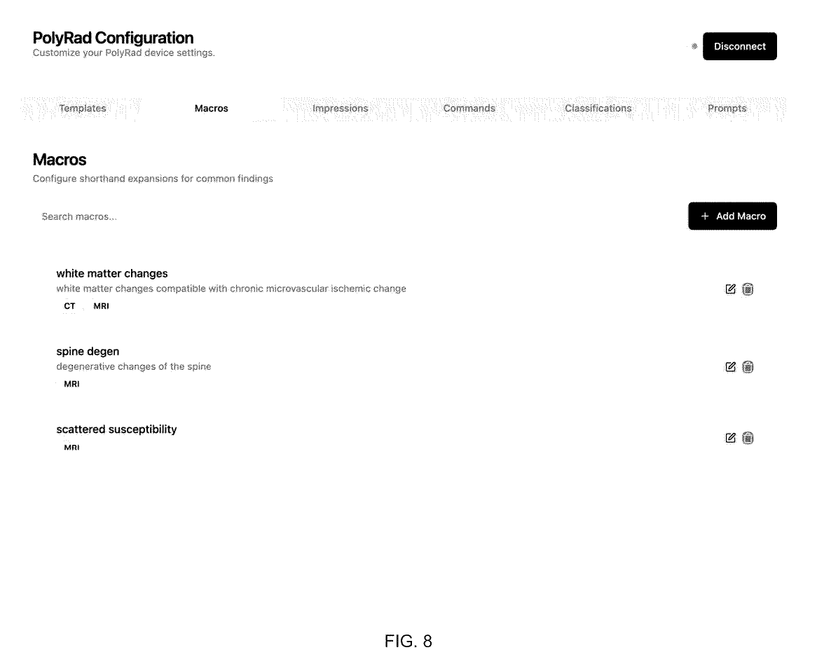

FIG. 8 is a screenshot showing one embodiment of a user interface of the software application. As shown in FIG. 8, the user interface may comprise a macros screen.

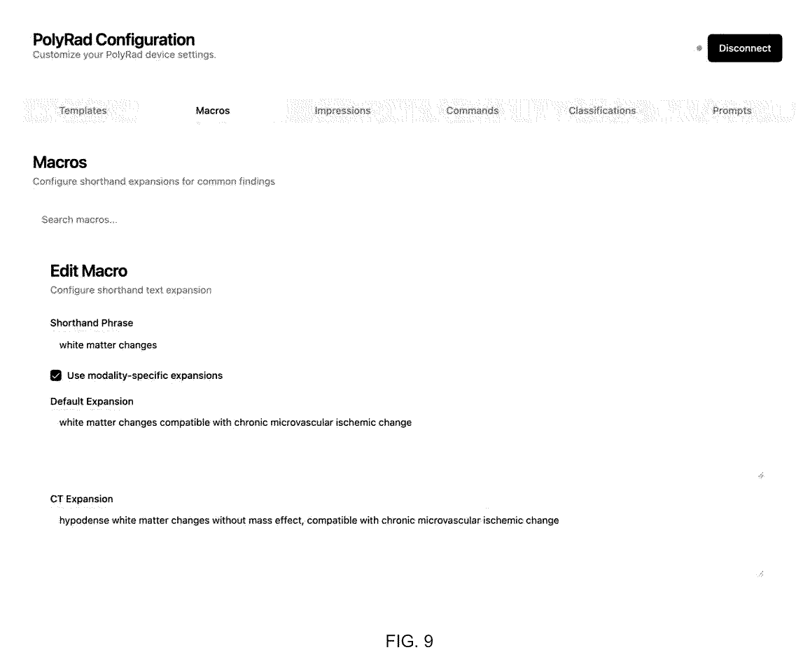

FIG. 9 is a screenshot showing one embodiment of a user interface of the software application. As shown in FIG. 9, the user interface may comprise an edit macros screen.

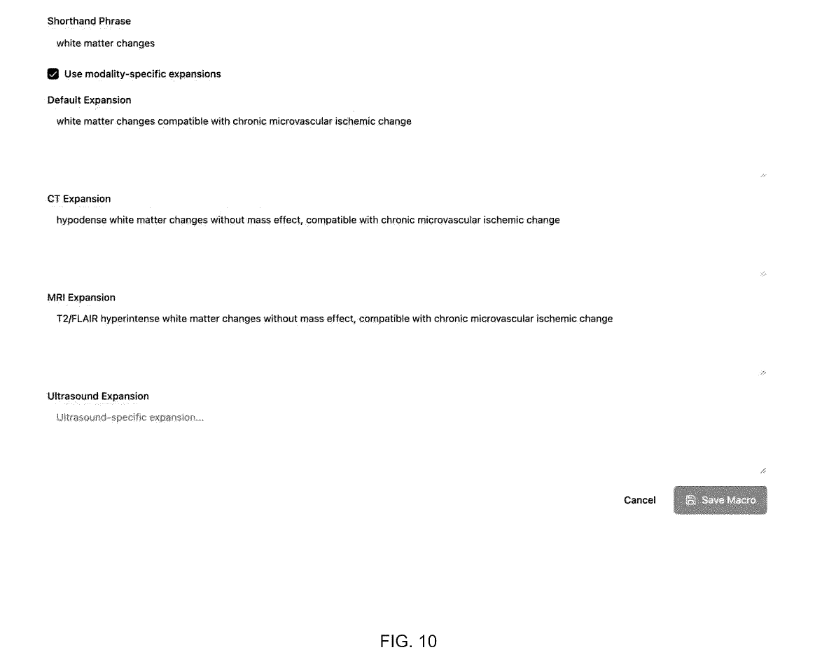

FIG. 10 is a screenshot showing one embodiment of a user interface of the software application. As shown in FIG. 10, the user interface may comprise an edit macros screen.

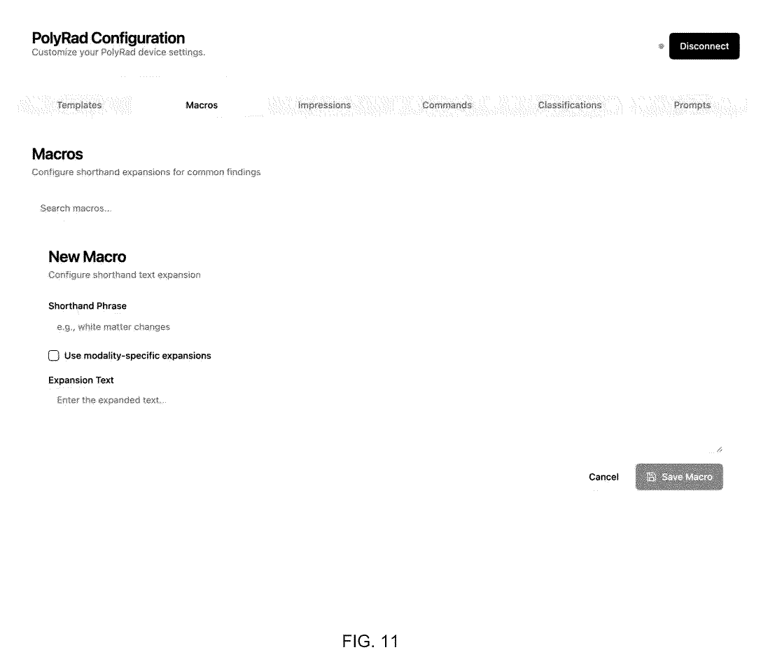

FIG. 11 is a screenshot showing one embodiment of a user interface of the software application. As shown in FIG. 11, the user interface may comprise a new macros screen.

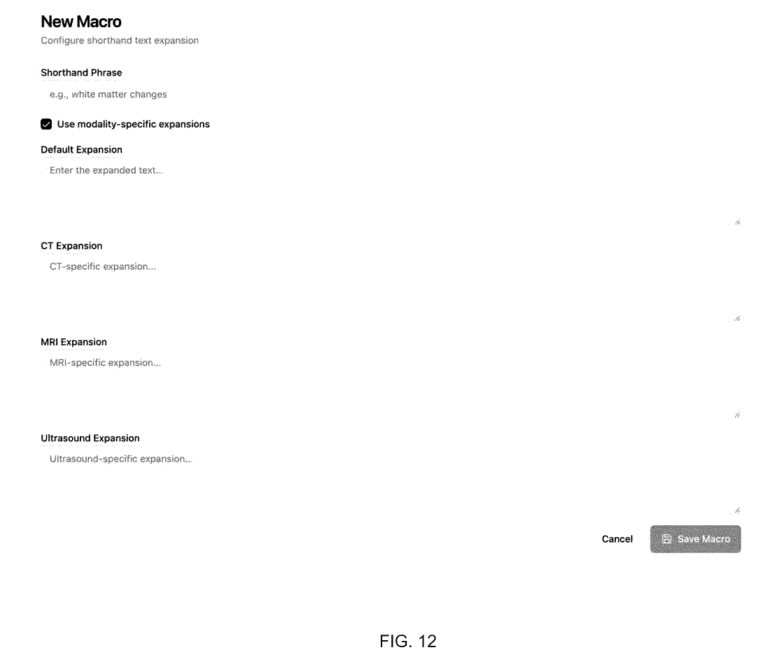

FIG. 12 is a screenshot showing one embodiment of a user interface of the software application. As shown in FIG. 12, the user interface may comprise a new macros screen and the save button.

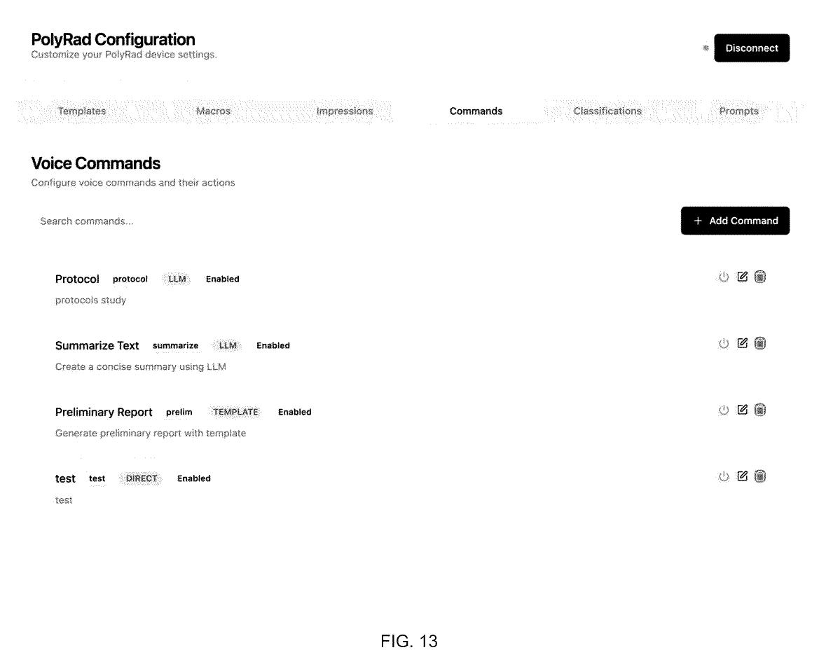

FIG. 13 is a screenshot showing one embodiment of a user interface of the software application. As shown in FIG. 13, the user interface may comprise a voice commands screen.

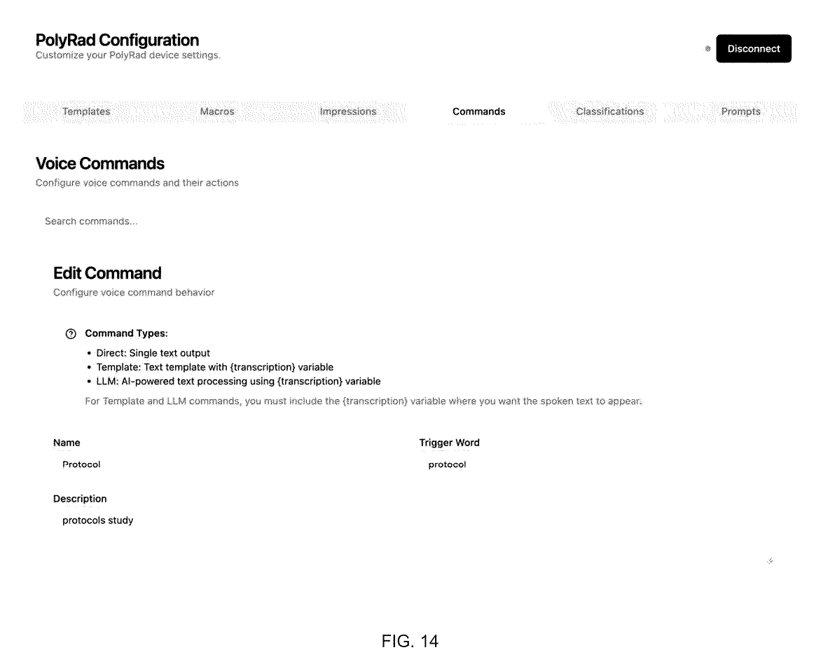

FIG. 14 is a screenshot showing one embodiment of a user interface of the software application. As shown in FIG. 14, the user interface may comprise an editing voice commands screen.

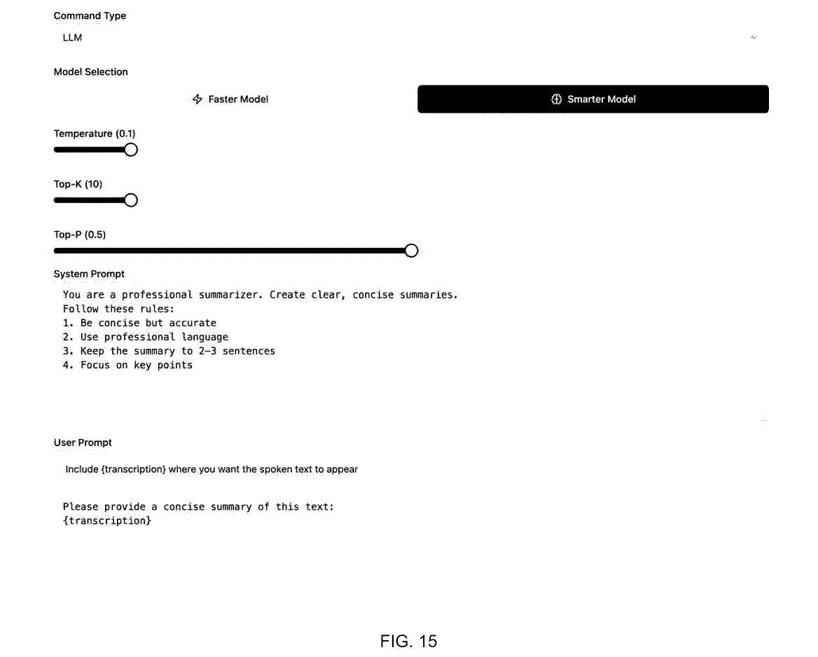

FIG. 15 is a screenshot showing one embodiment of a user interface of the software application. As shown in FIG. 15, the user interface may comprise an LLM prompt screen.

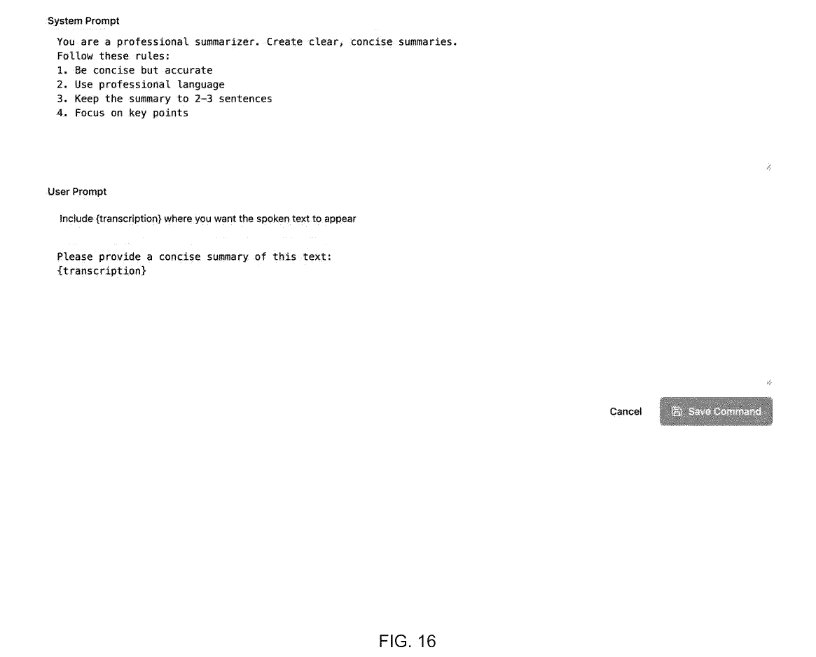

FIG. 16 is a screenshot showing one embodiment of a user interface of the software application. As shown in FIG. 16, the user interface may comprise a system and user prompt screen.

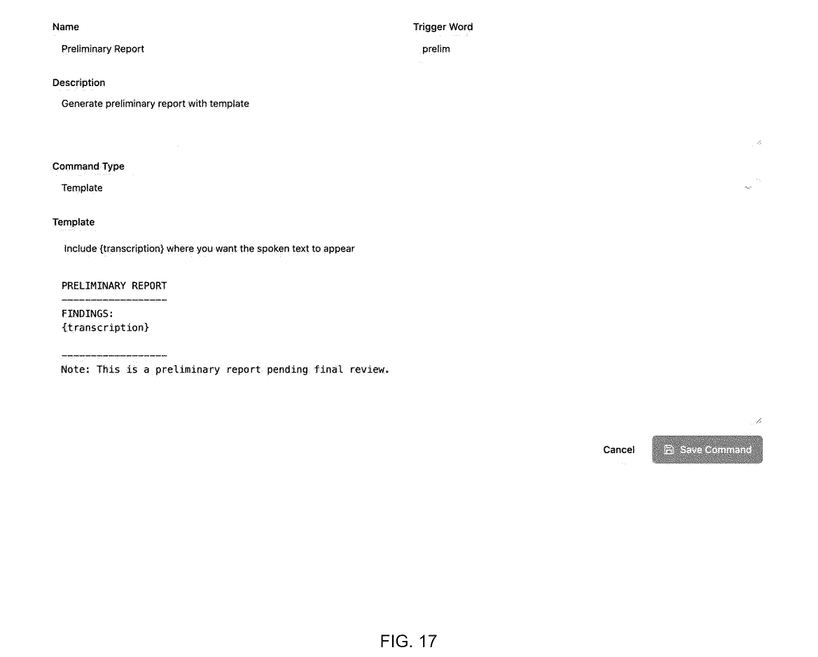

FIG. 17 is a screenshot showing one embodiment of a user interface of the software application. As shown in FIG. 17, the user interface may comprise a saving command screen.

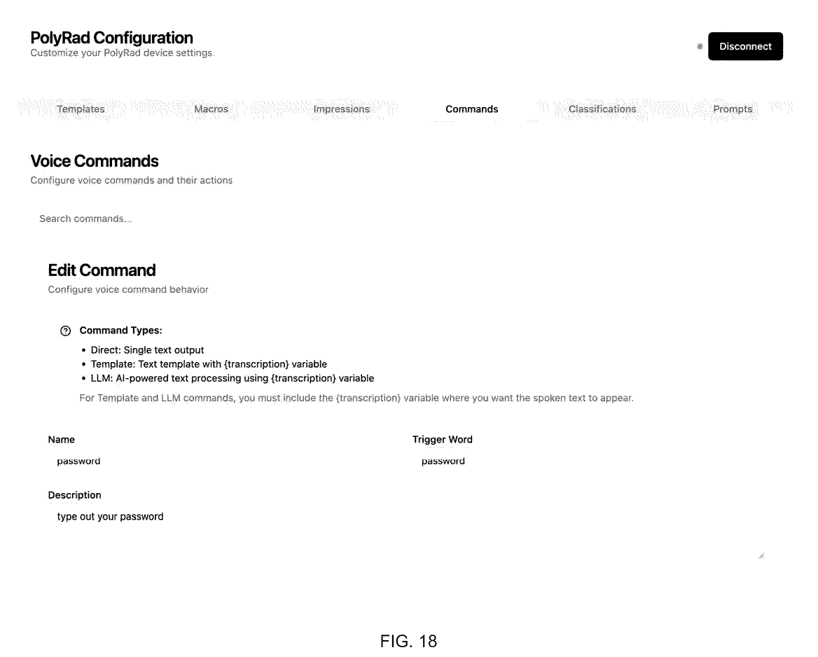

FIG. 18 is a screenshot showing one embodiment of a user interface of the software application. As shown in FIG. 18, the user interface may comprise an editing voice commands screen.

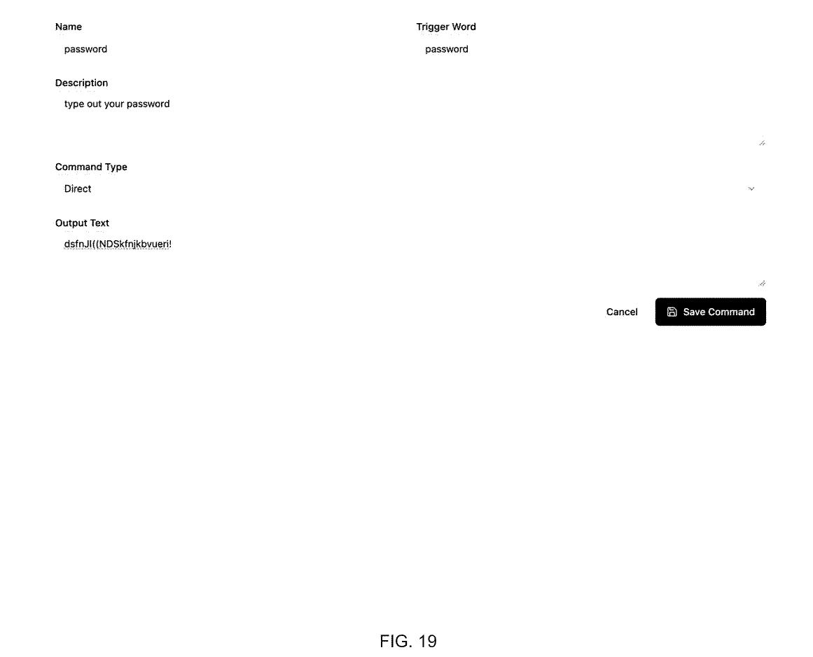

FIG. 19 is a screenshot showing one embodiment of a user interface of the software application. As shown in FIG. 19, the user interface may comprise a saving voice commands screen.

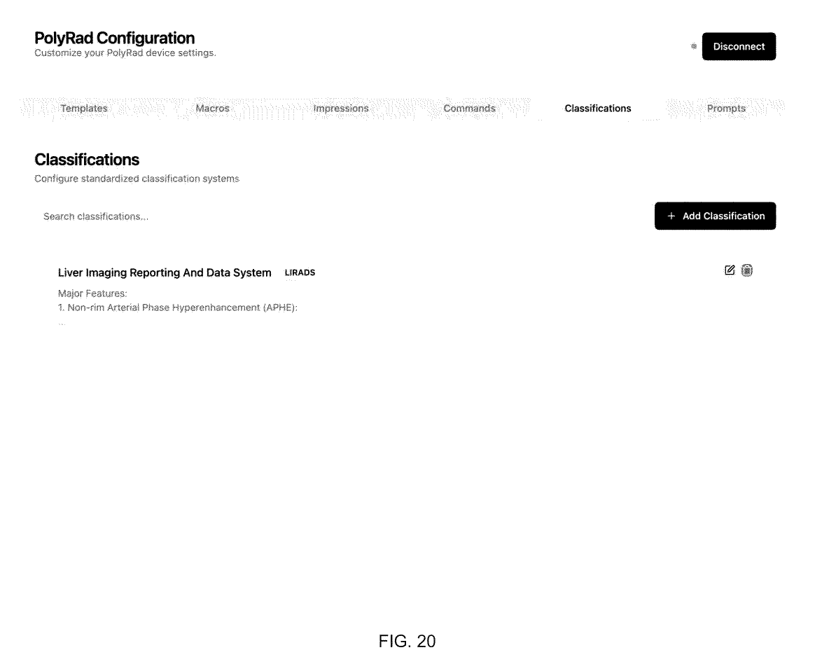

FIG. 20 is a screenshot showing one embodiment of a user interface of the software application. As shown in FIG. 20, the user interface may comprise a classifications screen.

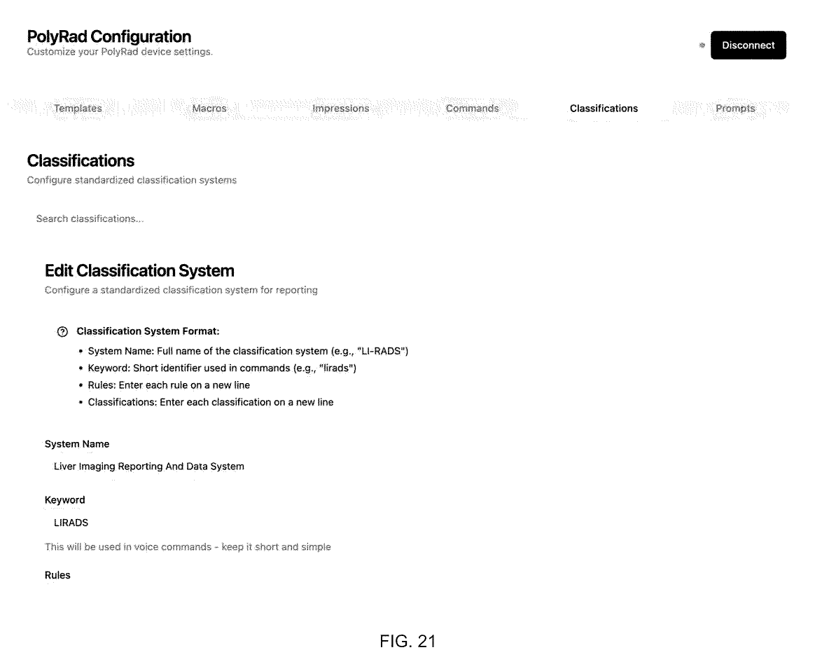

FIG. 21 is a screenshot showing one embodiment of a user interface of the software application. As shown in FIG. 21, the user interface may comprise an editing classifications screen.

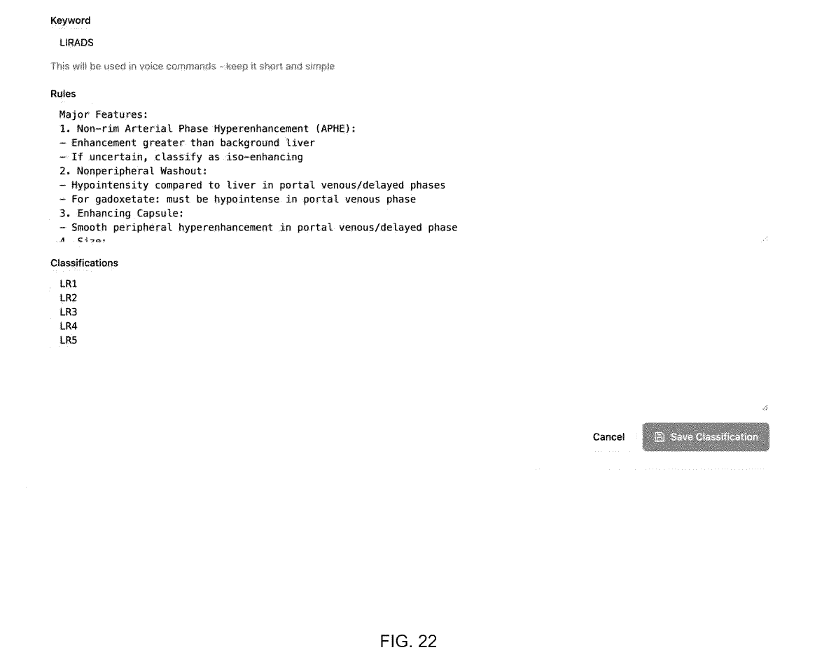

FIG. 22 is a screenshot showing one embodiment of a user interface of the software application. As shown in FIG. 22, the user interface may comprise a saving classifications screen.

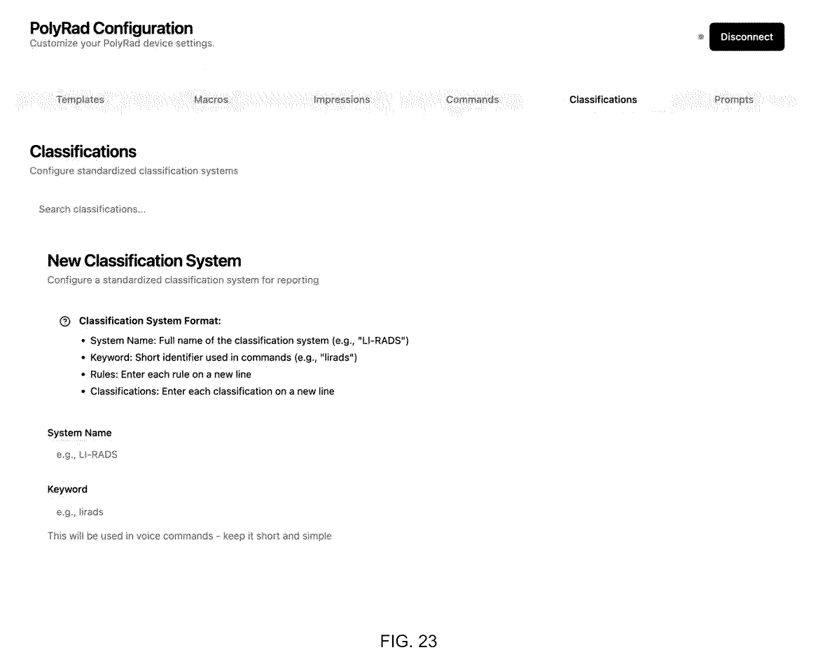

FIG. 23 is a screenshot showing one embodiment of a user interface of the software application. As shown in FIG. 23, the user interface may comprise a prompt pipeline screen.

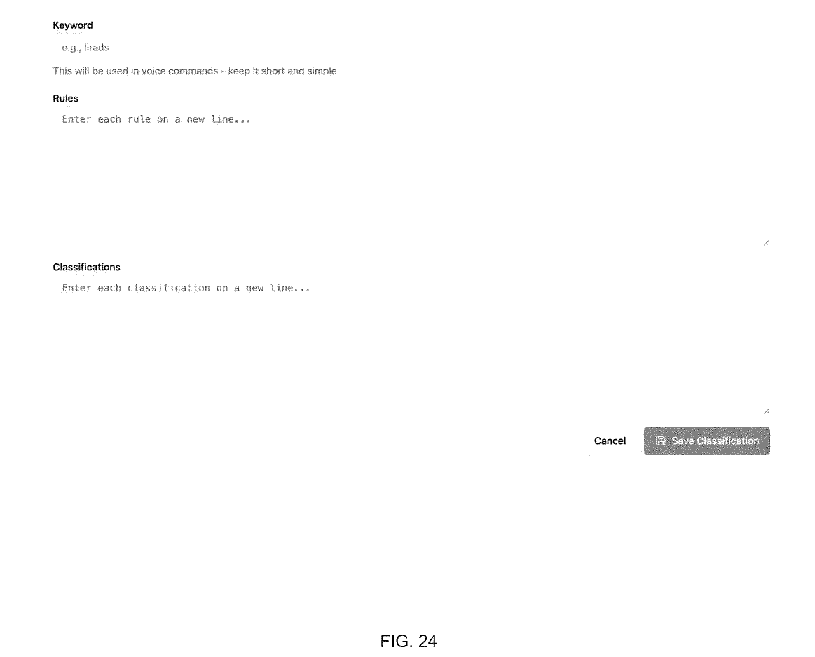

FIG. 24 is a screenshot showing one embodiment of a user interface of the software application. As shown in FIG. 24, the user interface may comprise an editing prompt screen.

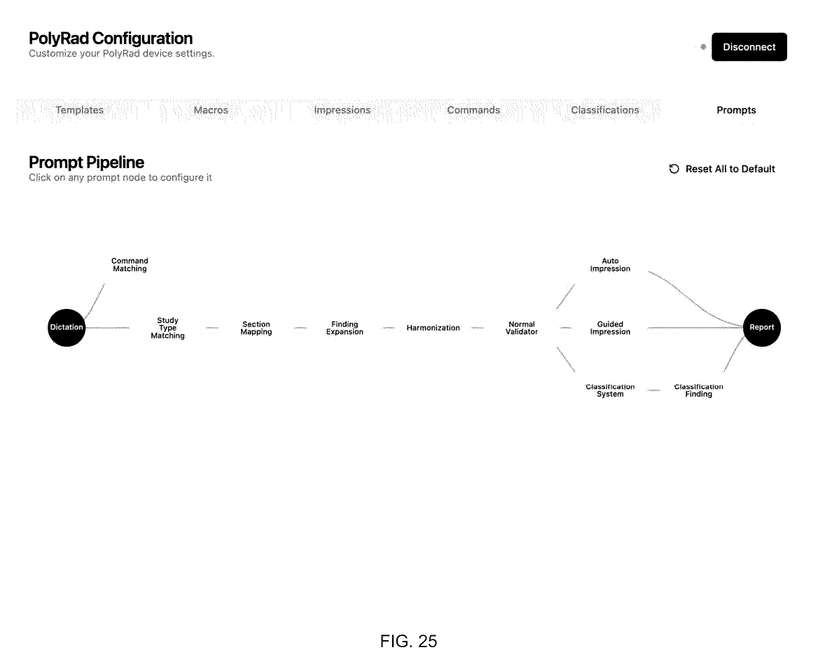

FIG. 25 is a screenshot showing one embodiment of a user interface of the software application. As shown in FIG. 25, the user interface may comprise a saving prompt screen.

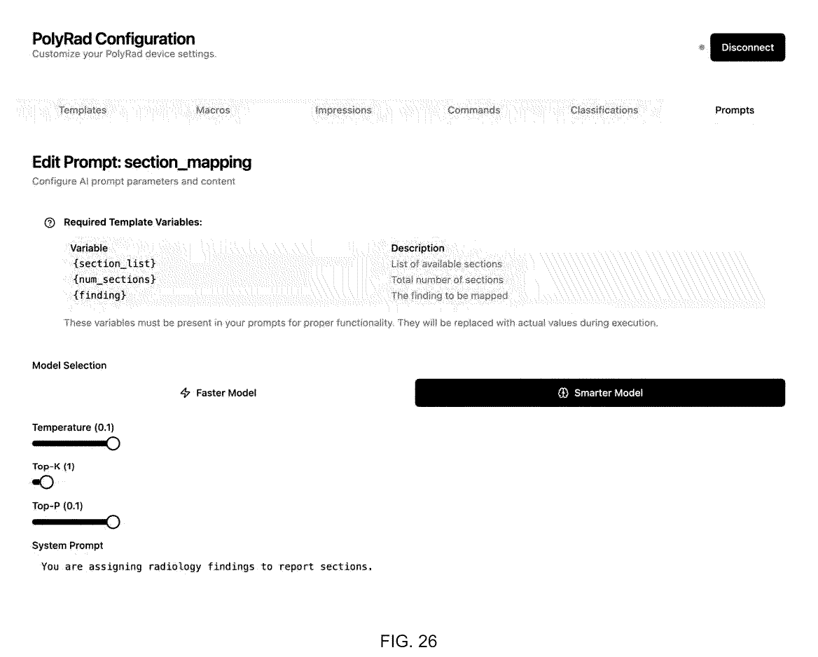

FIG. 26 is a screenshot showing one embodiment of a user interface of the software application. As shown in FIG. 26, the user interface may comprise a configuring the AI for a prompt screen.

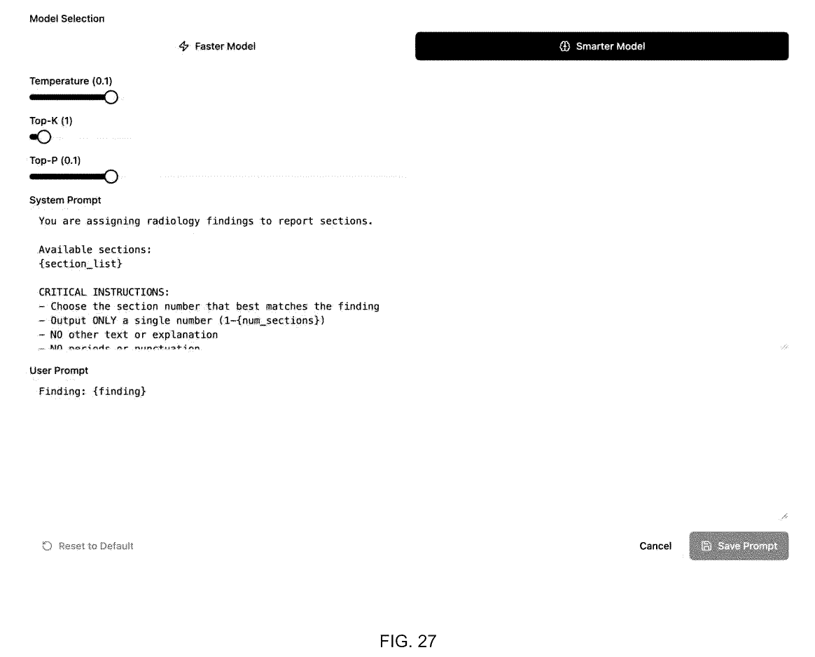

FIG. 27 is a screenshot showing one embodiment of a user interface of the software application. As shown in FIG. 27, the user interface may comprise an editing the AI for a prompt screen.

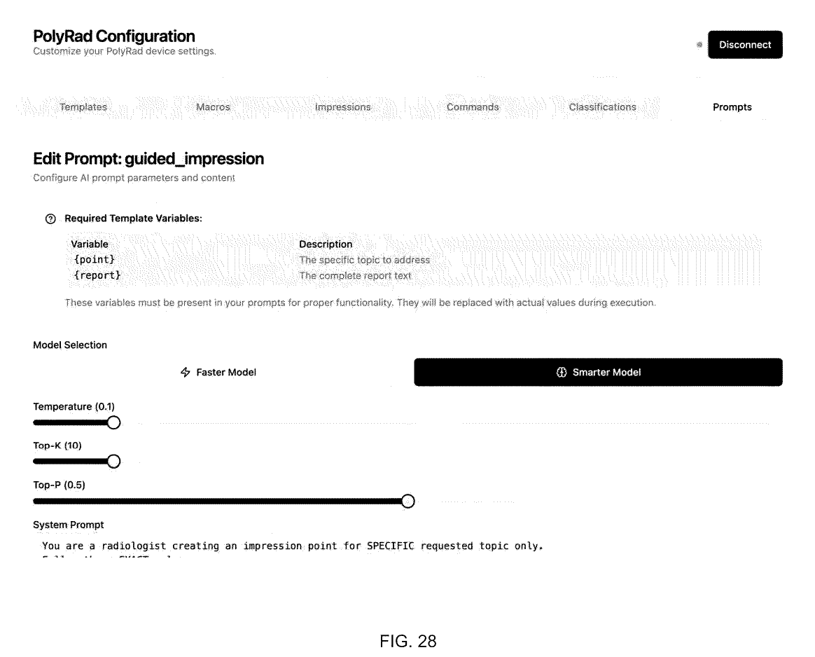

FIG. 28 is a screenshot showing one embodiment of a user interface of the software application. As shown in FIG. 28, the user interface may comprise an editing the AI for a prompt screen.

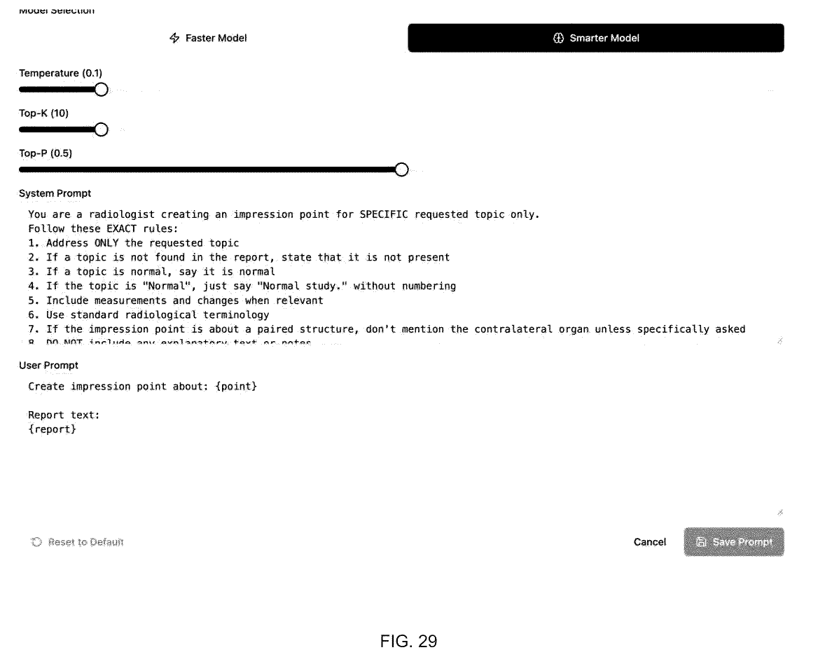

FIG. 29 is a screenshot showing one embodiment of a user interface of the software application. As shown in FIG. 29, the user interface may comprise an editing the AI for a prompt screen.

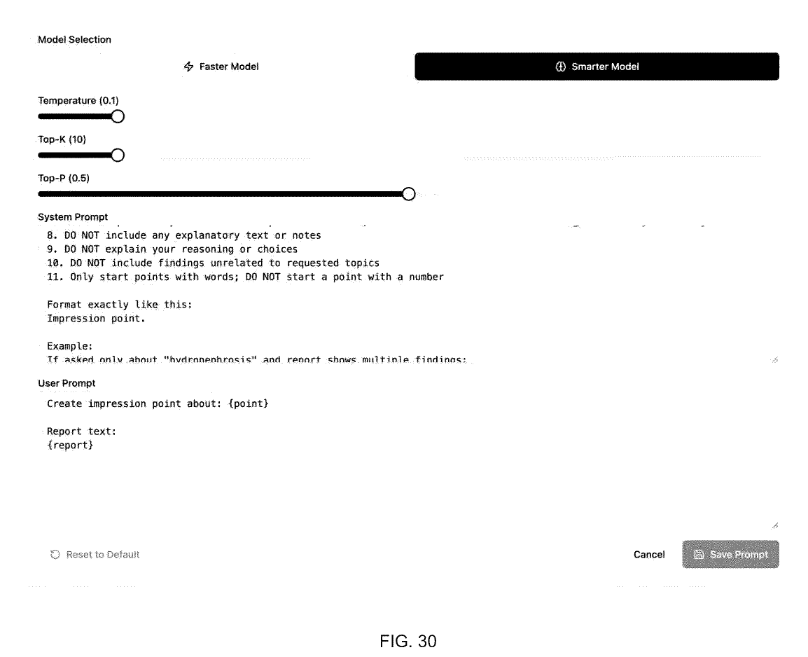

FIG. 30 is a screenshot showing one embodiment of a user interface of the software application. As shown in FIG. 30, the user interface may comprise an editing the AI for a prompt screen.

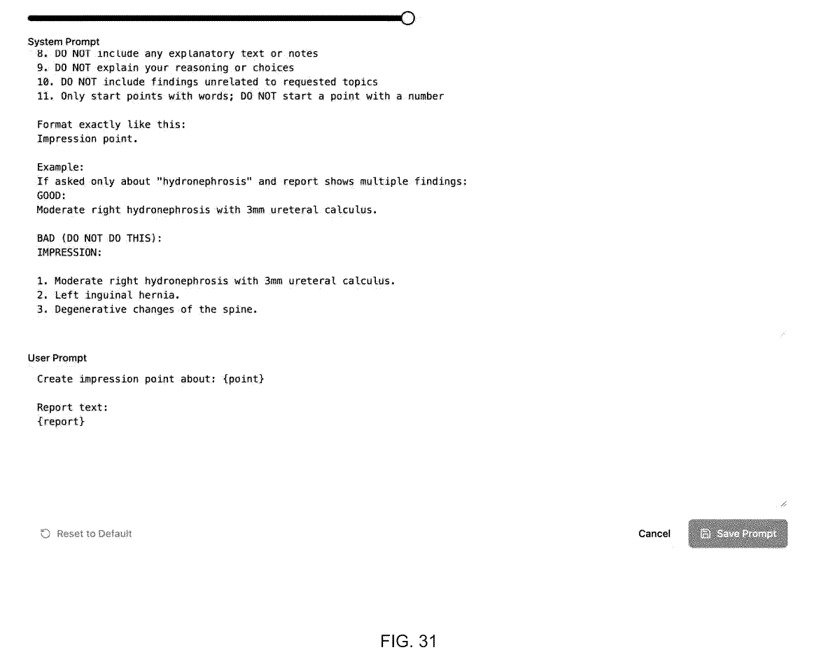

FIG. 31 is a screenshot showing one embodiment of a user interface of the software application. As shown in FIG. 31, the user interface may comprise an editing the AI for a prompt screen.

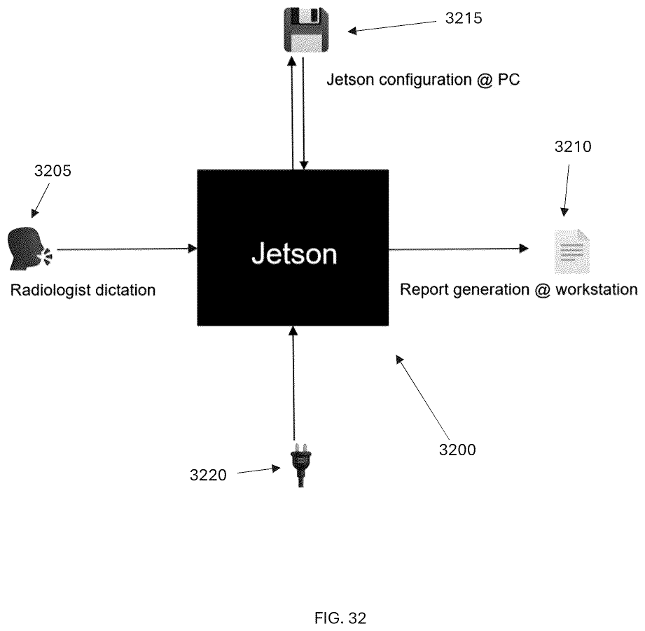

FIG. 32 is a flow block diagram of one embodiment of a system for simulating keyboard input. As shown in FIG. 32, device 3200 may receive verbal/audio dictation from user 3205 and generate a report at workstation 3210. The device 3200 may also send and receive information, such as configurations and macros from a separate computer 3215. In some embodiments, the device 3200 may receive power from an external power source 3220. In other embodiments, the device 3200 may have an internal power source, such as a replaceable or rechargeable battery, or receive power from separate computer 3215 or workstation 3210.

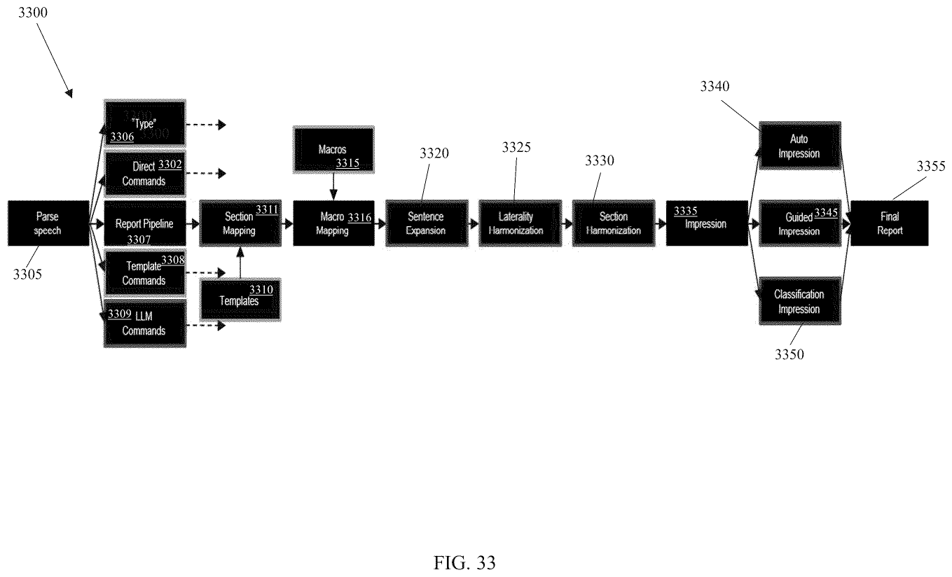

FIG. 33 is a flow diagram of one embodiment of a system and method for processing speech of the present disclosure. As shown in FIG. 33, the first step in process 3300 may be to parse speech 3305, by determining speech type 3306 and speech direct commands 3302. The speech is also parsed through template commands 3308 and LLM commands 3309, and put into the report pipeline 3307. The next step may be Section Mapping 3311, which may use templates provided by or selected from Templates 3310. If desired the user may prepare or be provided with Macros 3315, which may be processed into Macro Mapping 3316. The next step may be further processing of the speech, including, Sentence Expansion 3320, Laterality Harmonization 3325, and Section Harmonization 3330, which may be performed by different LLMs or the same LLM. The speech may, optionally, be further processed by Impression 3335 LLM, which may be one or more of Auto Impression 3340 LLM, Guided Impression 3345 LLM, and Classification Impression 3350 LLM. The final step of the pipeline/method is the production of Final Report 3355.

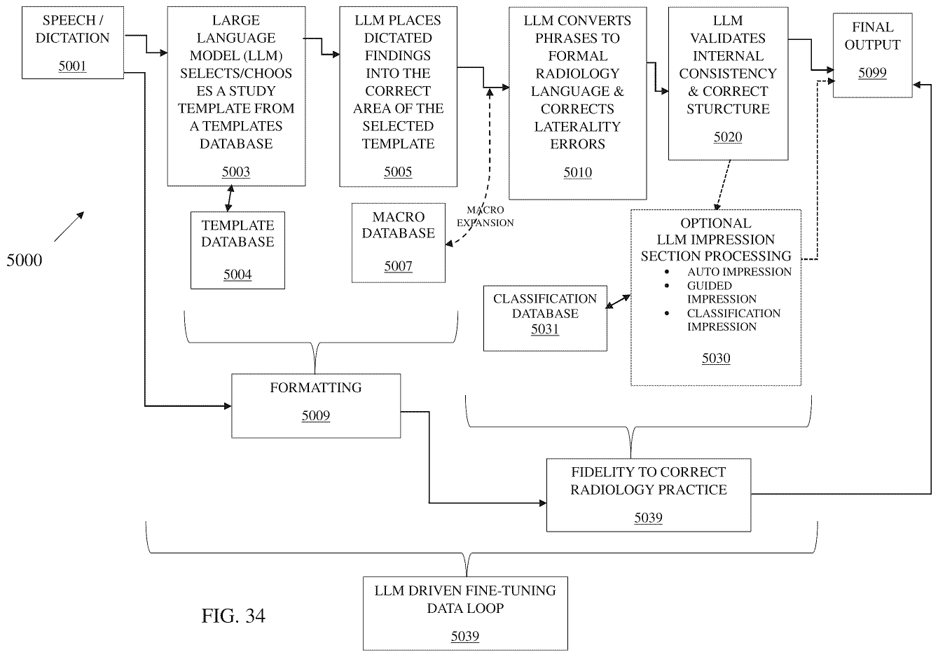

FIG. 34 is a flow block diagram of one embodiment of a system and method for processing speech to simulate keyboard input. The system may primarily comprise device 100 detailed in FIG. 1. Device 100 may be part of process 5000, which starts with intaking speech or dictation 5001 and ends with a report or final output 5099. In broad terms this process may be referred to as LLM driven fine-tuning data loop 5039, which may have two generalized components (other than input 5001 and output 5099), formatting 5009 and fidelity to correct radiology practice 5039. The formatting 5009 may preferably include (1) LLM selecting or choosing a study template 5003 from template database 5004 and (2) LLM dictated findings are positioned into the correct area of the selected template 5005. If desired, between formatting 5009 and fidelity to correct radiology practice 5039, the user may select macro expansion via macro database 5007. The fidelity to correct radiology practice 5039 may include (1) LLM converting phrases to formal radiology language and correcting laterality errors 5010 (example, right kidney instead of left kidney) and (2) LLM validating for internal consistency and correct structure 5020. Optionally, there may be LLM impression section processing 5030, which may be one of three types: (a) auto impression (no guidance from the user); (b) guided impression (where the user mandates what is included); and (c) classification impression (which is guided impression enhanced by LLM determination of a defined radiological classification (provided by classification database 5031), such as, but not limited to, LI-RADS (Liver Imaging Reporting and Data System), TI-RADS (Thyroid Imaging Reporting and Data System), and AAST Guide (American Association for the Surgery of Trauma).

Prior to the process shown in FIG. 34, a voice-to-text LLM (“A”) may start the process by converting voice to text. Then, as shown in FIG. 34, a small/fast/less intelligent LLM (“B”), and a larger/slower/more intelligent LLM (“C”) may be used at various points shown in FIG. 34. Each step may utilize B or C as customized or preferred by the user. In other embodiments, the functionality of B and C may be combined into a single LLM. Various embodiments may utilize different LLMs that are configured to function at different levels.

Some portions of the preceding detailed descriptions have been presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and, generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it should be appreciated that throughout the present disclosure, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display devices.

The processes or methods depicted in the figures may be performed by processing logic that comprises hardware (e.g., circuitry, dedicated logic, etc.), firmware, software (e.g., embodied on a non-transitory computer readable medium), or a combination thereof. Although the processes or methods are described above in terms of some sequential operations, it should be appreciated that some of the operations described may be performed in a different order. Moreover, some operations may be performed in parallel rather than sequentially.

In addition, the various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize that many modifications may be made to this configuration without departing from the scope of the disclosed embodiments.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded as illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A device for simulating keyboard input, comprising:
an electronic processing unit;
a microcontroller;
one or more IO ports; and
a software application;
wherein said one or more IO ports comprises a first IO port;
wherein said first IO port is configured to connect to a computer;
wherein said software application comprises one or more large language models (“LLM”); and
wherein said microcontroller is configured to generate one or more keystrokes and output said one or more keystrokes through said first IO port.

2. The device of claim 1, wherein said one or more LLMs comprise a first LLM, a second LLM, and a third LLM.

3. The device of claim 2, wherein said first LLM comprises a speech to text protocol;
wherein said second LLM comprises a fast output protocol; and
wherein said third LLM comprises a refined output protocol.

4. The device of claim 3, wherein said software application is configured to receive an audio.

5. The device of claim 4, wherein said first LLM is configured to convert said audio to a transcription text;
wherein said second LLM is configured to convert said transcription text to a fast report text; and
wherein said third LLM is configured to convert said fast report text to a refined report text.

6. The device of claim 5, wherein said one or more keystrokes are generated based on said refined report text.

7. The device of claim 6, wherein said software application is configured to receive one or more instructions.

8. The device of claim 7, wherein said one or more instructions comprise one or more macros, such that when said transcription text, said report text, or said refined report text is generated, the respective text is modified pursuant to said one or more macros.

9. The device of claim 7, wherein said one or more instructions comprise one or more macros, such that when said transcription text, said report text, or said refined report text is generated, the respective text includes modifications to said one or more macros.

10. The device of claim 7, wherein said one or more LLMs are configured to incorporate said one or more instructions into their respective processing steps.

11. The device of claim 5, further comprising a microphone configured to receive said audio.

12. The device of claim 5, wherein said one or more IO ports comprise a second IO port.

13. The device of claim 12, wherein said second IO port is configured to receive said audio from a microphone.

14. The device of claim 5, further comprising an activator configured to allow said audio to be processed.

15. The device of claim 14, wherein said activator is a button.

16. The device of claim 1, wherein said first IO port is wireless.

17. The device of claim 1, wherein said first IO port is wired.

18. The device of claim 1, wherein said electronic processing unit is a graphics processing unit.

19. The device of claim 1, wherein said electronic processing unit is a central processing unit.

20. A device for simulating keyboard input, comprising:
an electronic processing unit;
a microcontroller;
one or more IO ports;
a microphone;
an activator; and
a software application;
wherein said one or more IO ports comprises a first IO port;
wherein said first IO port is configured to connect to a computer;
wherein said software application comprises one or more large language models (“LLM”);
wherein said microcontroller is configured to generate one or more keystrokes and output said one or more keystrokes through said first IO port;
wherein said software application is configured to receive an audio;
wherein said one or more LLMs comprise a first LLM, a second LLM, and a third LLM;
wherein said first LLM comprises a speech to text protocol;
wherein said second LLM comprises a fast output protocol;
wherein said third LLM comprises a refined output protocol;
wherein said first LLM is configured to convert said audio to a transcription text;
wherein said second LLM is configured to convert said transcription text to a fast report text;
wherein said third LLM is configured to convert said fast report text to a refined report text;
wherein said one or more keystrokes are generated based on said refined report text;
wherein said software application is configured to receive one or more instructions; and
wherein said one or more instructions comprise one or more macros, such that when said transcription text, said fast report text, or said refined report text is generated, the respective text is modified pursuant to said one or more macros.
